(19) United States
(12) Patent Application Publication
(10) Pub. No.: US 2022/0362532 A1
(43) Pub. Date: Nov. 17, 2022

(54) APPLICATOR DEVICE AND METHODS OF USE

(71) Applicant: Janine HOPKINS, Monroe, LA (US)

(72) Inventor: Janine HOPKINS, Monroe, LA (US)

(21) Appl. No.: 17/771,703

(22) PCT Filed: Oct. 23, 2020
(86) PCT No.: PCT/US2020/057151

§ 371 Date: Apr. 25, 2022

Related U.S. Application Data
(60) Provisional application No. 62/925,527, filed on Oct. 24, 2019.

(51) Int. Cl. A61M 37/00; A61M 5/31

(52) U.S. Cl. CPC A61M 37/0015; A61M 5/3134; A61M 2202/0415; A61M 2202/045; A61M 2202/0427; A61M 2210/04

(57) ABSTRACT

The present invention is directed to an applicator, configured to reversibly attach to a dispensing device, with at least one micropore and a kit for administration of a therapeutic agent. Methods of use of the applicator are also disclosed herein.

This application is an International Application, which claims the benefit of priority from U.S. Provisional Patent Application No. 62/925,527, filed on Oct. 24, 2019, the entire content of which are incorporated herein by reference in its entirety.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

Not applicable.

### FIELD OF THE INVENTION

The present invention is directed to a device, a kit, and associated methods for controlled application of a therapeutic solution.

### SUMMARY OF THE INVENTION

In one aspect, the present invention provides devices for controlled application of a therapeutic solution to a tissue of a subject in need thereof. In this aspect, the device comprises an applicator. The applicator can further comprise at least one micropore. In embodiments, the size of the at least one micropore is configured to maintain the integrity of a component within the therapeutic solution during application. The at least one micropore can comprise an opening that is substantially flush with a bottom surface of the applicator. The device can further include a means for reversibly attaching the applicator to a dispensing device. In one embodiment, the dispensing device comprises a syringe.

In certain embodiments, the dispensing device comprises a container of commercially available topical therapeutic agent, and the therapeutic solution comprises the contents of the container of commercially available topical therapeutic agent. In such embodiments, the means for reversibly attaching the applicator to the container of commercially available topical therapeutic agent comprises a first threaded surface disposed on a neck of the applicator. The first threaded surface can comprise threads that are complementary to a second threaded surface that is disposed near an opening of the container of commercially available topical therapeutic agent.

In embodiments, the device comprises a plurality of micropores. Each of the micropores can be substantially identical in size. In certain embodiments, the therapeutic solution comprises a topical solution.

The device can be configured for use with a cosmetic procedure, a medical procedure, or a combination thereof. In some embodiments, the medical procedure comprises cancer treatment, lesion treatment, pain control, wound care, burn care, skin care, or a combination thereof. The cosmetic procedure can comprise treatment of skin conditions, treatment of skin damage, skin resurfacing, skin rejuvenation, or a combination thereof

In embodiments, the means for attaching the applicator to a dispensing device comprises a Luer fitting.

In various embodiments, the therapeutic solution comprises a therapeutic agent. The therapeutic agent can comprise a topical cream. In certain embodiments, the topical cream comprises an antibiotic, an analgesic, a steroid, an anti-fungal agent, or a combination thereof. The therapeutic agent can comprise a pharmaceutical composition, a biological fluid, or a combination thereof. In embodiments, the biological fluid is autologous, homologous, heterologous, or a combination thereof. The biological fluid can comprise serum, stem cells, a platelet concentrate, or a combination thereof. Exemplary platelet-rich concentrates include platelet-rich plasma (PRP), platelet-rich fibrin (PRF), or a combination thereof.

The diameter of at least one micropore can be any diameter at least the size of the inner diameter of a 33-gauge needle. At least one micropore can comprise a diameter that is at least about 0.1 mm. In embodiments, at least one micropore comprises a diameter that is less than about 0.5 mm. The micropore can comprise a diameter of about 0.1 to 0.2 mm. In embodiments, at least one micropore comprises a diameter of about 1 mm or less. In certain embodiments, the plurality of micropores comprises at least four micropores. The plurality of micropores can comprise up to 20 micropores.

In embodiments, the bottom surface of the applicator is substantially circular, substantially oval, or polygonal in shape. The applicator can be substantially disc-shaped.

The applicator can comprise a medical-grade material. In certain embodiments, the applicator comprises a surgical metal., a medical-grade polymer, or a combination thereof. The surgical metal can comprise stainless steel, titanium, tantalum, gold, platinum, palladium, or a combination thereof.

In various embodiments, the tissue comprises skin, muscle, connective tissue, or a combination thereof. The tissue can comprise wounded, fractionated or resurfaced skin. In embodiments, the tissue comprises dermis, epidermis, subcutaneous fat, fascia, or a combination thereof.

The device can be reusable or disposable.

One embodiment includes a device for controlled application of a platelet concentrate during a cosmetic resurfacing procedure, wherein the device comprises a disc-shaped applicator. The disc-shaped applicator can further comprise at least 12 micropores, wherein each of the micropores comprise a diameter of 1 mm or less. Each of the micropores can include an opening that is substantially flush with a bottom surface of the applicator. Embodiments further include a Luer fitting. In embodiments, the platelet-rich concentrate comprises platelet-rich plasma (PRP), platelet-rich fibrin (PRF), or a combination thereof.

Disclosed in another aspect is a method of administering one or more therapeutic solutions to a tissue of a subject in need thereof. In embodiments, the method comprises obtaining a volume of therapeutic solution and expressing the volume of therapeutic solution through the device in accordance with any of the embodiments described in this disclosure and onto the tissue of the subject. In embodiments, the integrity of the therapeutic solution is not affected by expression therethrough. In embodiments, the therapeutic solution comprises a biological fluid, and obtaining the volume of therapeutic solution comprises drawing the biological fluid into a syringe. The method can further include attaching the applicator to the syringe. In certain embodiments, the therapeutic solution comprises a topical therapeutic agent. The dispensing device can comprise a container of commercially available topical therapeutic agent. In an embodiment, the method further comprises attaching the applicator to the container of commercially available topical therapeutic agent.

Another aspect includes a method of reducing contamination during application of a topical solution to a subject in need thereof, comprising obtaining a volume of topical solution; and expressing the volume of topical solution through the device in accordance with any of the embodiments described in this disclosure and onto the tissue of the subject, wherein the integrity of the topical solution is not affected by expression therethrough.

Yet another aspect discloses a kit for applying a therapeutic solution. In embodiments, the kit comprises a device in accordance with any of the embodiments described in this disclosure and instructions for use. In certain embodiments, the kit includes a sterile syringe, a therapeutic agent, or a combination thereof.

An additional aspect includes a kit for treating or restoring a tissue of a subject comprising the device in accordance with any of the embodiments described in this disclosure and instructions for use.

In another aspect, a kit for use with cosmetic resurfacing procedures is disclosed wherein the kit comprises the device in accordance with any of the embodiments described in this disclosure and instructions for use.

Other objects and advantages of this invention will become readily apparent from the ensuing description.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 provides a schematic side view of an applicator in accordance with an embodiment of the present invention.

FIG. 2 shows schematic bottom view of an applicator in accordance with one embodiment of the present invention. The bottom of surface of the applicator is shown with fourteen pores or channels extending therethrough.

FIG. 3 provides a schematic, a top view of an applicator in accordance with an embodiment of the present invention.

FIG. 4 provides a photographic view of an exemplary means for attachment of the applicator to a dispensing device under one embodiment.

FIG. 5A shows a photographic top view of an applicator in accordance with one embodiment of the present invention.

FIG. 5B shows a schematic top view of an applicator in accordance with another embodiment of the present invention.

FIG. 6A provides a configuration for a circle-shaped bottom surface under one embodiment of the present invention.

FIG. 6B provides a configuration for a circle-shaped bottom surface under another embodiment of the present invention.

FIG. 6C provides a configuration for a circle-shaped bottom surface under an alternate embodiment of the present invention.

FIG. 6D shows a configuration for a sunburst-shaped bottom surface under one embodiment of the present invention.

FIG. 6E provides a configuration for a square-shaped bottom surface with rounded edges under one embodiment.

FIG. 7A provides a schematic side view of an applicator in accordance with an alternate embodiment of the present invention.

FIG. 7B provides a schematic side view of an applicator in another embodiment of the present invention.

FIG. 8A shows a top perspective view of an applicator under one embodiment.

FIG. 8B provides a schematic side view of the FIG. 8A embodiment.

FIG. 8C shows top schematic view of the FIG. 8A embodiment.

FIG. 8D shows a schematic cross-sectional view of the FIG. 8A embodiment. The view is shown through cross-section 385 of FIG. 8C

FIG. 8E provides a bottom schematic view of the FIG. 8A embodiment.

FIG. 8F provides a view through the FIG. 8E cross-section 387 of the bottom surface 301.

FIG. 8G shows a close-up view of the micropores from section 389 of FIG. 8F.

FIG. 9A shows a top perspective view of an applicator under another embodiment.

FIG. 9B provides a schematic side view of the FIG. 9A embodiment.

FIG. 9C shows a schematic cross-sectional view of the FIG. 9B embodiment.

FIG. 9D provides a bottom schematic view of the FIG. 9A embodiment.

FIG. 10A shows a top perspective view of an applicator under another embodiment.

FIG. 10B provides a schematic side view of the FIG. 10A embodiment.

FIG. 10C shows a schematic cross-sectional view of the FIG. 10B embodiment.

FIG. 11 provides a bottom perspective view of an applicator under one embodiment of the present invention attached to a dispensing device.

FIG. 12A shows a side view of an applicator under one embodiment of the present invention. A bottom portion of the applicator is shown in phantom to reveal a plurality of through openings therein.

FIG. 12B shows a bottom view of the FIG. 12A applicator.

FIG. 13A provides a side view of an applicator under another embodiment of the present invention. A bottom portion of the applicator is shown in phantom to reveal a plurality of through openings therein. The applicator is shown attached to a dispensing device.

FIG. 13B shows a bottom view of the FIG. 13A applicator.

FIG. 14 provides a graphical representation of flow deviation through each of the micropores of the FIG. 12 embodiment. Measurements were obtained in the three cases described in Table 1, and each numeral represents a different micropore or nozzle.

FIG. 15 provides a graphical representation of flow deviation through each of the micropores of the FIG. 13 embodiment. Measurements were obtained in the three cases described in Table 1, and each numeral represents a different micropore or nozzle.

FIG. 16 shows a schematic view taken from within the body and looking down on a bottom portion of an applicator in yet another embodiment of the present invention.

FIG. 17A provides a graphical representation of flow rate deviation as water passes through each of the micropores of the FIG. 16 embodiment at a flow rate of 0.5 ml/sec. Each numeral represents a different micropore or nozzle.

FIG. 17B provides a graphical representation of flow rate deviation as water passes through each of the micropores of the FIG. 16 embodiment at a flow rate of 1 ml/sec. Each numeral represents a different micropore or nozzle.

FIG. 17C provides a graphical representation of flow rate deviation as petroleum jelly (heated to 100° C.) passes through each of the micropores of the FIG. 16 embodiment at a flow rate of 0.5 ml/sec. Each numeral represents a different micropore or nozzle.

FIG. 18A shows a side view of the bottom portion of an applicator under one embodiment of the present invention.

FIG. 18B provides a side, cross-sectional view of the FIG. 18A bottom portion.

FIG. 18C shows the bottom surface and micropore configuration of the FIG. 18A embodiment.

FIG. 19A provides a graphical representation of flow rate deviation as water passes through each of the micropores of the FIG. 18 embodiment at a flow rate of 0.5 ml/sec. Each numeral corresponds to the applicable micropore or nozzle of FIG. 18C.

FIG. 19B provides a graphical representation of flow rate deviation as water passes through each of the micropores of the FIG. 18 embodiment at a flow rate of 1 ml/sec. Each numeral corresponds to the applicable micropore or nozzle of FIG. 18C.

FIG. 19C provides a graphical representation of flow rate deviation as petroleum jelly (heated to 100° C.) passes through each of the micropores of the FIG. 18 embodiment at a flow rate of 0.5 ml/sec. Each numeral corresponds to the applicable micropore or nozzle of FIG. 18C.

FIG. 20A provides a graphical snapshot of fluid dynamics at 2 milliseconds following initiation of water dispersion at a flow rate of 0.5 ml/second (T=2 ms). In the large, central portion of the figure, the applicator and through openings (also referred to herein as “nozzles”) are shown in phantom and water is shown filled within the applicator neck and body. The inset provides a cross-sectional view of the applicator at T=2 ms.

FIG. 20B shows graphical snapshot of fluid dynamics at about 8 milliseconds following initiation of water dispersion at a flow rate of 0.5 ml/second (T=8 ms). In the large, central portion of the figure, the applicator and nozzles are shown in phantom and fluid is shown passing from the body and into the nozzles. The inset provides a cross-sectional view of the applicator at T=8 ms.

FIG. 20C shows graphical snapshot of fluid dynamics at about 56 milliseconds following initiation of water dispersion at a flow rate of 0.5 ml/second (T=56 ms). In the large, central portion of the figure, the applicator and nozzles are shown in phantom and fluid is shown passing exiting the nozzles of the applicator. The inset provides a cross-sectional view of the applicator at T=56 ms.

FIG. 21A provides a schematic side view of an applicator under an embodiment with a disc-shaped body.

FIG. 21B shows a top perspective view of the FIG. 21A embodiment.

FIG. 21C shows bottom view of the FIG. 21A embodiment.

FIG. 21D shows a bottom perspective view of the FIG. 21A embodiment.

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” refers to “wherein the lever extends substantially vertically” so long as a precise vertical arrangement is not necessary for the lever to perform its function.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” can refer to the process including at least steps a, b and c. Wherever the terms “a” or “an” are used, “one or more” is understood, unless such interpretation is nonsensical in context.

The term “about” is used herein to mean approximately, roughly, around, or in the region of When the term “about” is used in conjunction with a numerical range, it modifies that range by extending the boundaries above and below the numerical values set forth. For example, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The terms “subject” and “patient” as used herein can include all members of the animal kingdom including, but not limited to, mammals, animals (e.g., cats, dogs, horses, swine, etc.) and humans.

The term “tissue” as used herein can refer to any conglomeration of cells along with the extracellular matrix that work in concert to carry out a specific function. In embodiments, tissue includes nervous tissue, epithelial tissue, connective tissue, muscular tissue, or a combination thereof. Tissue can include dermis, epidermis, subcutaneous fat, fascia, or any combination thereof. In certain embodiments, the tissue may be injured or diseased. Injured or diseased tissue can refer to any tissue that is inflamed, dry, cancerous, wounded, abraded, eroded, burned, fractionated, or has been subjected to any other type of tissue injury or disease or combinations thereof. Injured or diseased tissue can refer to any of various skin conditions known in the art.

The term “therapeutic agent” can refer to any material or substance or combination of materials or substances that includes at least one therapeutic compound that is suitable for use in any medical or cosmetic procedure. Therapeutic agent can also include a cosmeceutical agent. Therapeutic agents can exist in liquid form. In various exemplary embodiments, the therapeutic agent can include biological fluids, pharmaceutical compositions, or any other therapeutic compound known by one of skill in the art. A therapeutic agent can comprise an antimicrobial agent. In certain embodiments, the therapeutic agent is suspended within a solution. The therapeutic agent can be suspended in a solution configured for topical application.

A “therapeutic solution” can mean any solution that comprises a therapeutic agent.

As used herein, the term “medical procedure” can mean any course of action that is intended to achieve a desired result in the field of healthcare. In certain embodiments, medical procedures can include cancer treatment, lesion treatment, pain control, wound care, burn care, skin care, or a combination thereof. Medical procedures can comprise treatment of any skin condition or rash known in the art. Skin conditions can comprise itchy, red, dry, irritated, or raised skin. The skin condition can be a bacterial or fungal infection. Non-limiting examples of skin conditions comprises dermatophytosis, eczema, diaper rash, seborrheic dermatitis, chickenpox, psoriasis, rosacea, measles, warts, acne, fifth disease, or a combination thereof. Lesions can include cancerous or precancerous lesions. Non-limiting examples of pre-cancerous lesions include actinic keratosis, actinic cheilitis. Examples of cancerous lesions include but are not limited to melanoma, basal cell carcinoma, or squamous cell carcinoma. The term “medical procedure” can further encompass any procedure used in a research setting to study a health-related condition or to study or develop treatments for a health-related condition.

As used herein, the term “cosmetic procedure” can mean any procedure intended to alter the physical appearance of a subject or a subject's tissue. In embodiments, a cosmetic procedure includes any aesthetic procedure. Cosmetic procedures can be non-invasive or minimally invasive procedures. Cosmetic procedures include any procedure known by one of skill in art to be a cosmetic or aesthetic procedure. Exemplary cosmetic procedures include but are not limited to treatment of skin conditions, treatment of skin damage, skin resurfacing, skin rejuvenation, or a combination thereof. In embodiments, cosmetic procedures include treatment of skin laxity, wrinkles, scars, skin discoloration or pigmentation changes, acne scars, cellulite, visible blood vessels or other vascular conditions, or combinations thereof. In embodiments, skin discoloration or pigmentation includes liver spots, moles, freckles, sunspots, or other forms of skin discoloration known in the art.

Topical therapeutic agents are often applied via a syringe and needle. This is true when the topical therapeutic agent comprises a biological fluid. For instance, an emerging treatment for skin rejuvenation involves the use of platelet-rich plasma (PRP) obtained via a venipuncture followed by centrifugation to harvest the blood-borne product. PRP is then transferred to a sterile syringe from which it can be injected into the skin or applied to skin that have been treated with fully ablative or fractional skin resurfacing. Currently, there is no medical device on the market to deliver PRP with controlled application to the skin. Needles or an open syringe are used to express the product from the syringe onto the skin. The risks include needle sticks, splatter, and contamination of the PRP product.

The use of a syringe and needles introduces a heightened risk of contamination and needle sticks. Expression of the fluid through the needle can occur at a high rate of speed, causing splash-back of the effluent and any biological material therein, which may spread contaminants that can cause infection or transmit disease. In addition, use of needles can be associated with unnecessary needle sticks to the subject, the persons administering the topical solution, or those assisting with the procedure. High pressure expression through a narrow needle can also disrupt the integrity of material, such as platelets, within the therapeutic agent.

Topical agents, such as those purchased over-the-counter or received according to prescription are also applied in an unsanitary manner that risks exposure of the caretaker, the subject, or the product to contaminants or infections biological material. For instance, over-the-counter topical ointments such as antimicrobials, antibiotics, anti-viral agents, analgesics, corticosteroids, anti-fungal agents, and the like are applied by first expressing the topical ointment onto the caretaker's non-sterile bare or gloved finger and then applying the ointment directly to the area of treatment. This treatment method risks contamination of the product being applied, for example, when the ointment is applied multiple times to the same treatment site. This further risks introduction of contaminants or other irritants into the treatment site that could exacerbate or worsen the subject's condition. Finally, direct application of the ointment risks exposure of the caretaker to biological material or contaminants that can cause infection or transmit disease.

In various exemplary embodiments, the presently disclosed applicator is configured to permit safe, controlled and sterile application of a therapeutic agent, such as PRP and topical ointments, to the skin reduce the aforementioned common problems with administration of topical solutions. This apparatus can be configured to allow practitioners to apply PRP to the skin safely while maintaining the integrity of the PRP particles. The presently disclosed applicator reduces the risk of both contamination and needlesticks by removing the necessity of applying a therapeutic agent via a needle attached to syringe. In embodiments, the apparatus can be configured to permit safe, sterile application of therapeutic agents such as ointments at home by a caretaker. The applicator can be configured for use of a healthcare provider in a clinical setting.

In embodiments, the presently disclosed applicator is configured to reversibly attach to a dispensing device, such as a syringe or ointment dispenser, and comprises at least one micropore that permits controlled expression of a therapeutic agent with no needles, protuberances, or other extensions. The applicator can comprise a plurality of micropores for controlled delivery of the therapeutic agent in a manner that preserves the integrity of any materials disposed therein.

### Description of Selected Embodiments

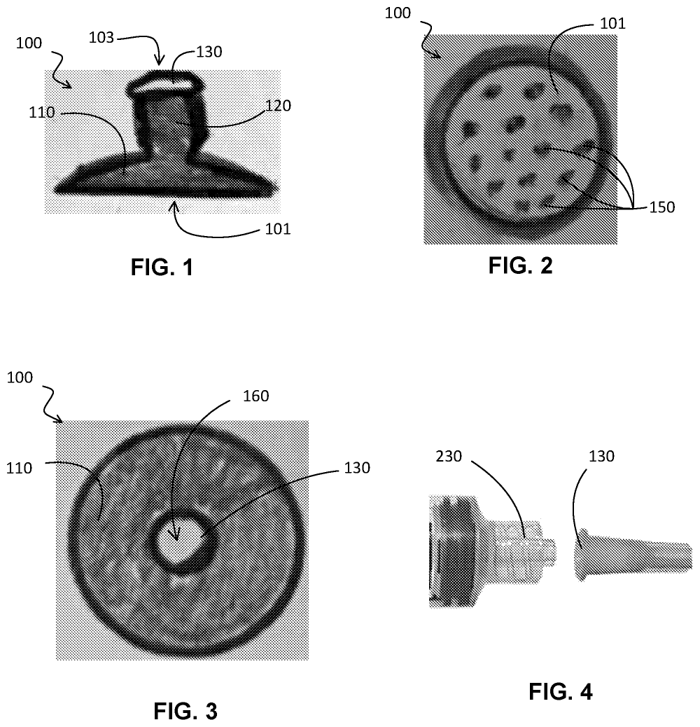

FIG. 1 provides a schematic, a side view of an applicator 100 in accordance with an embodiment of the present invention. As can be seen, in the present embodiment, the applicator 100 includes a top portion 103 and bottom surface 101. A means for reversibly attaching the applicator to a dispensing device 130 can be seen at the topmost portion of the applicator 100. In the FIG. 1 embodiment, the attachment means 130 is connected to a widened portion or body 110 of the applicator via a neck or collar 120. In embodiments, the body 110 forms a manifold with at least one through opening (seen more clearly at 150 of FIG. 2) that terminates in the bottom surface 101 of the applicator 100.

FIG. 2 shows the bottom surface 101 of the applicator 100 of FIG. 1. In the FIG. 2 embodiment, the bottom surface 101 is shown with fourteen openings 150 extending therethrough. In operation, the openings form micropores 150 that permit controlled application of a solution when the applicator 100 is connected to a dispensing device.

FIG. 3 provides a schematic, a top view of the applicator 100 of FIG. 1. The attachment means 130 can be seen as well as the body 110 of the applicator 100. In addition, an interior space or lumen 160 is visible through the opening of the attachment means 130. In embodiments, the applicator is hollow with the interior space or lumen 160 being continuous throughout the applicator 100.

FIG. 4 shows a Luer lock hub, which can serve as an attachment means 130 under one embodiment. In the FIG. 4 embodiment, the tip of a syringe is shown with a female Luer connection 230 which comprises a threaded hub, and the attachment means 130 comprises a male Luer fitting that is configured to reversibly attach to the female Luer connection 230 by screwing the male Luer fitting 130 thereto.

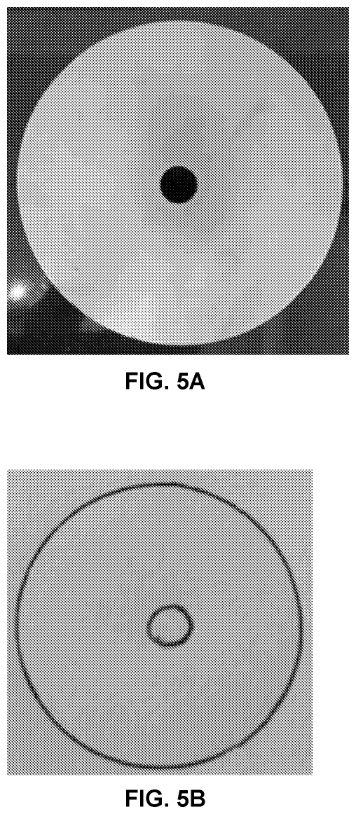

FIGS. 5A and 5B show top view of an applicator in accordance with one embodiment of the present invention.

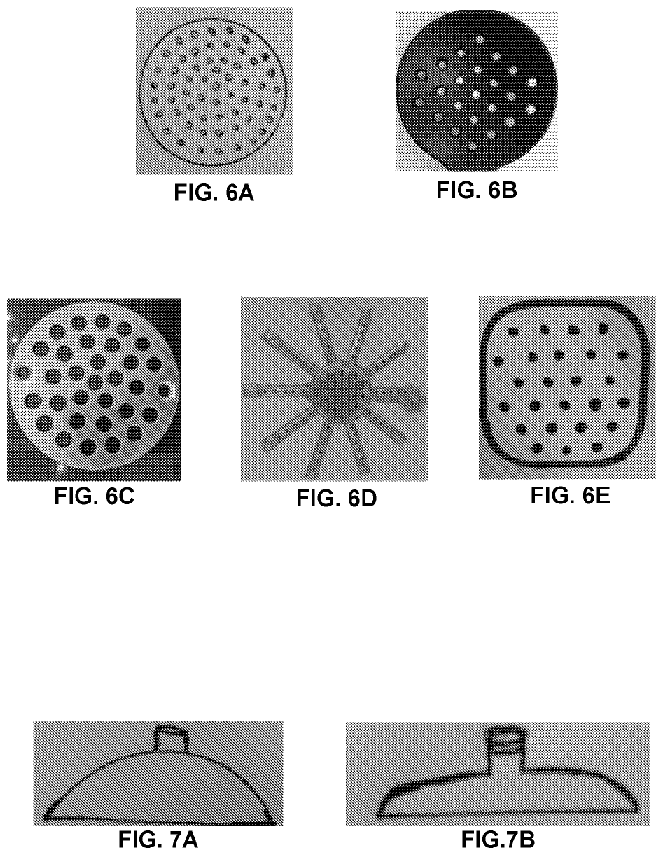

FIG. 6A-6E provide varying configurations for the bottom surface of the applicator under various exemplary embodiments.

FIGS. 7A & 7B provide schematic side views of applicators to reveal exemplary, non-limiting shapes of various alternate embodiments of the present invention.

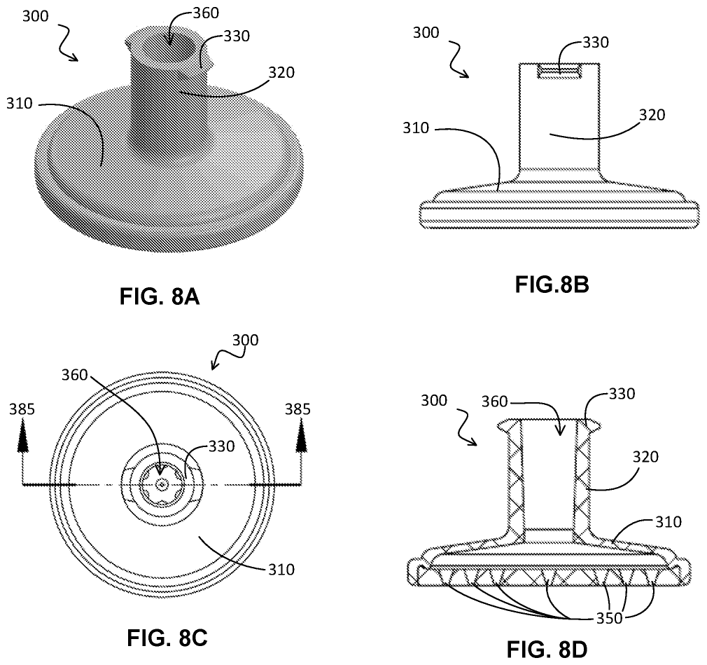

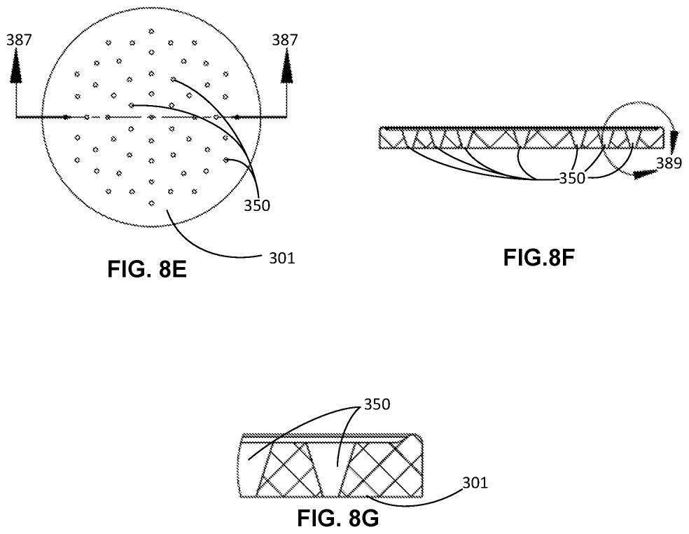

FIG. 8A shows a top perspective view of an applicator 300 under another embodiment. As can be seen, the applicator 300 includes a means for reversibly attaching the applicator to a dispensing device 330 at the topmost portion of the applicator 300. The attachment means 330 is connected to a widened portion or body 310 of the applicator via a neck or collar 320. This embodiment comprises a substantially disc-shaped body 310 that forms a manifold with at least one through opening (seen more clearly at 350 of FIG. 8D) that terminates in the bottom surface (seen more clearly at 301 of FIG. 8E) of the applicator 300.

FIG. 8B provides a schematic side view of the FIG. 8A embodiment 300.

FIG. 8C provides a schematic, a top view of the applicator 300 of FIG. 8A. The attachment means 330 can be seen as well as the body 310 of the applicator 300. In addition, an interior space or lumen 360 is visible through the opening of the attachment means 330.

FIG. 8D shows a schematic cross-sectional view of the FIG. 8A embodiment. The view is shown through cross-section 385 of FIG. 8C. As shown in this embodiment, the applicator 300 can be hollow with an interior space or lumen 360 being continuous throughout the applicator 300. The lumen 360 begins within the neck 320 of the applicator, extends into the body 310, and is continuous with the one or more through openings 350 that form micropores on the bottom surface 301 of the applicator.

FIG. 8E shows the bottom surface 301 of the applicator 300 of FIG. 8A. In the FIG. 8E embodiment, the bottom surface 301 is shown with 55 openings 350 extending therethrough. In operation, the openings form micropores 350 that permit controlled application of a solution or ointment when the applicator 300 is connected to a dispensing device.

FIG. 8F provides a view through the FIG. 8E cross-section 387 of the bottom surface 301. As can be seen, the micropores 350 in this embodiment comprise a conical shape which tapers as the opening exits the bottom surface of the applicator 300.

FIG. 8G shows a close-up view of the micropores from section 389 of FIG. 8F.

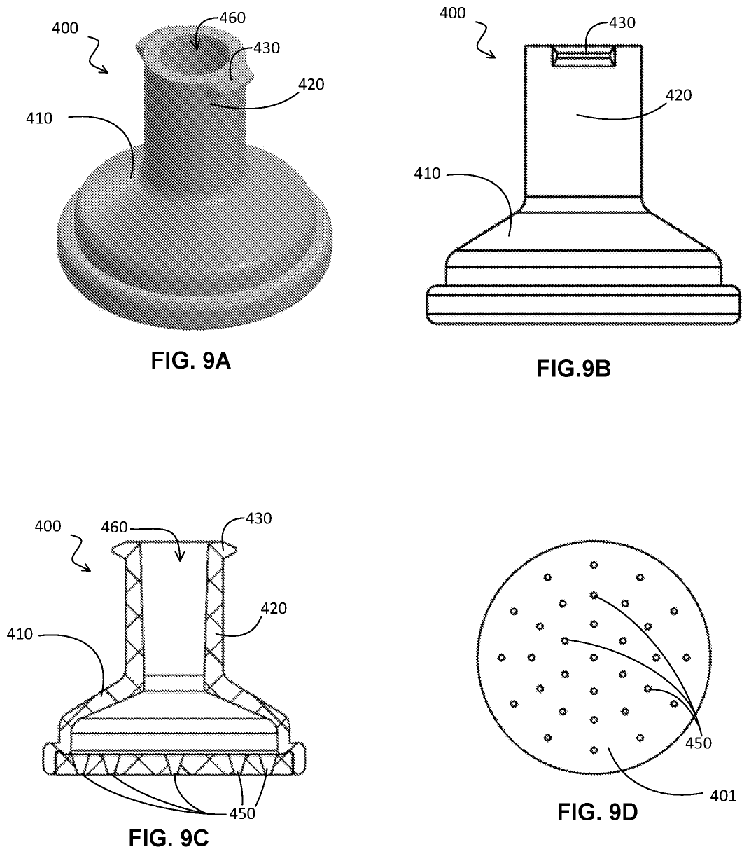

FIG. 9A shows a top perspective view of an applicator 400 under another embodiment. As can be seen, the applicator 400 includes a means for reversibly attaching the applicator to a dispensing device 430 at the topmost portion of the applicator 400. The attachment means 430 is connected to a widened portion or body 410 of the applicator via a neck or collar 420. This embodiment comprises a substantially dome-shaped body 410 that forms a manifold with at least one through opening (seen more clearly at 450 of FIG. 9C) that terminates in the bottom surface (seen more clearly at 401 of FIG. 9D) of the applicator 400.

FIG. 9B provides a schematic side view of the FIG. 9A embodiment 400.

FIG. 9C shows a schematic cross-sectional view of the FIG. 9A embodiment. As shown in this embodiment, the applicator 400 can be hollow with an interior space or lumen 360 being continuous throughout the applicator 400. The lumen 460 begins within the neck 420 of the applicator, extends into the body 410, and is continuous with the one or more through openings 450 that form one or more micropores on the bottom surface 401 of the applicator. As such, the lumen of the neck 420 is in fluid communication with the lumen of the body 410, which, in turn, is in fluid communication with the at least one through openings 450.

FIG. 9D provides a view of the bottom surface 401 of the applicator 400 of FIG. 9A. In the FIG. 9D embodiment, the bottom surface 401 is shown with 32 openings 450 extending therethrough. In operation, the openings form micropores 450 that permit controlled application of a solution or ointment when the applicator 400 is connected to a dispensing device.

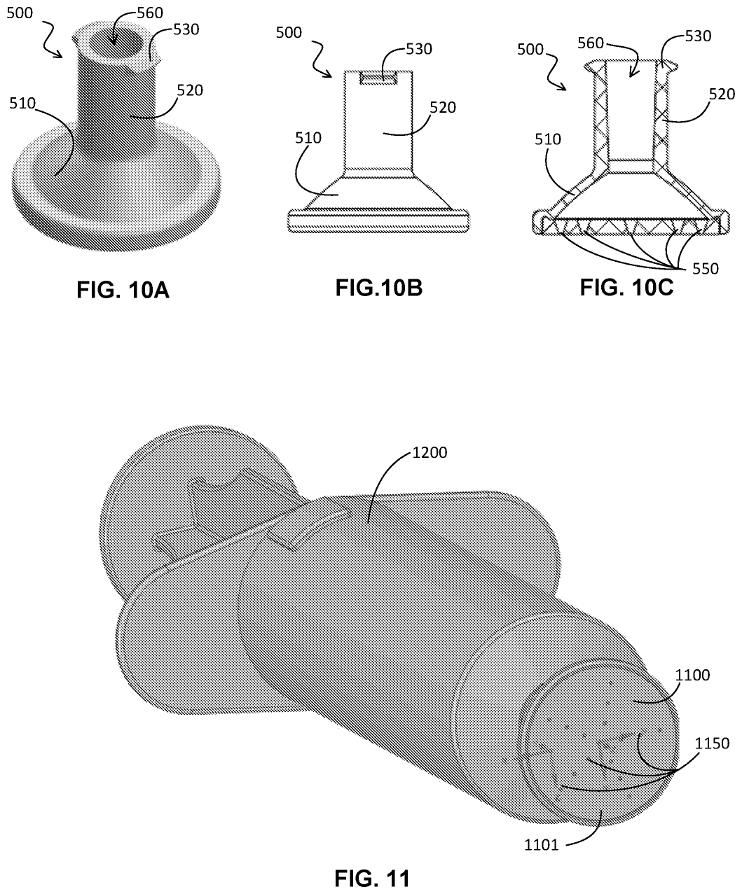

FIG. 10A shows a top perspective view of an applicator 500 under yet another embodiment. As can be seen, the applicator 500 includes a means for reversibly attaching the applicator to a dispensing device 530 at the topmost portion of the applicator 500. The attachment means 530 is connected to a widened portion or body 510 of the applicator via a neck or collar 520. This embodiment comprises a substantially dome-shaped body 510 that resembles an upside-down funnel design. The body 510 forms a manifold with at least one through opening (seen more clearly at 550 of FIG. 10C) that terminates in the bottom surface of the applicator 500.

FIG. 10B provides a schematic side view of the FIG. 10A embodiment 500.

FIG. 10C shows a schematic cross-sectional view of the FIG. 10A embodiment. As shown in this embodiment, the applicator 500 can be hollow with an interior space or lumen 560 being continuous throughout the applicator 500. The lumen 560 begins within the neck 520 of the applicator, extends into the body 510, and is continuous with the one or more through openings 550 that form one or more micropores on the bottom surface of the applicator. As such, the lumen of the neck 520 is in fluid communication with the lumen of the body 510, which, in turn, is in fluid communication with the at least one through openings 550.

As shown in FIGS. 8D, 8F, 8G, 9C, and 10C the through openings 350, 450, 550 can comprise a conical shape which tapers as the openings exit the bottom surface of the applicator to form micropores. As a result, in such embodiments, the through openings 350, 450, 550 are the narrowest at the point where the through openings 350, 450, 550 exit the bottom surface 301, 401, 501 of the applicator. In alternative embodiments, the through openings comprise an upside-down cone that becomes wider as the openings approach the bottom surface of the applicator to form the micropores. In these alternate embodiments, the through openings are widest at the point where the openings exit the bottom surface of the applicator. In still other embodiments, the through-openings are substantially cylindrical.

FIG. 11 provides a bottom perspective view of an applicator 1100 under one embodiment of the present invention attached to a dispensing device 1200. In the FIG. 11 embodiment, the dispensing device 1200 comprises a syringe, and depression of the syringe plunger forces the content of the syringe to pass through the barrel of the syringe and into the neck of the applicator. The contents then flow into the body of the applicator and exit the applicator through the plurality of micropores 1150 on the bottom surface of the syringe.

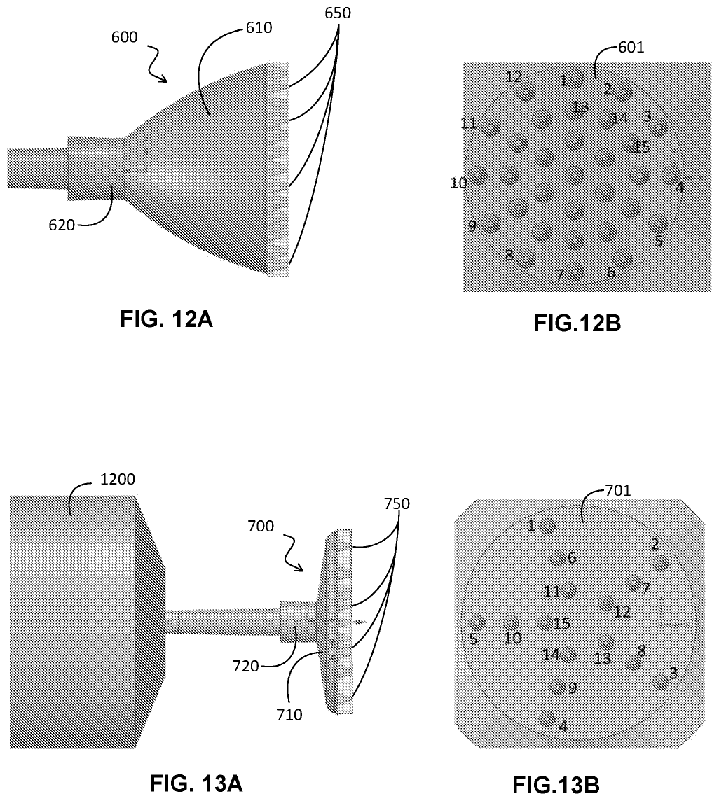

FIG. 12A shows a side view of an applicator 600 under an additional embodiment of the present invention. As can be seen, the applicator 600 in this embodiment comprises a deep dome-shaped body 610 that extends from the neck 620. In the FIG. 12A embodiment, a bottom portion of the applicator is shown in phantom to reveal through openings 650 in the form of nozzles 650 that extend from the body 610 of the applicator 600 and terminate as micropores on the bottom surface 601.

FIG. 12B shows the bottom surface 601 of the FIG. 12A applicator 600. In the pictured embodiment, the applicator 600 comprises 31 individual through-openings.

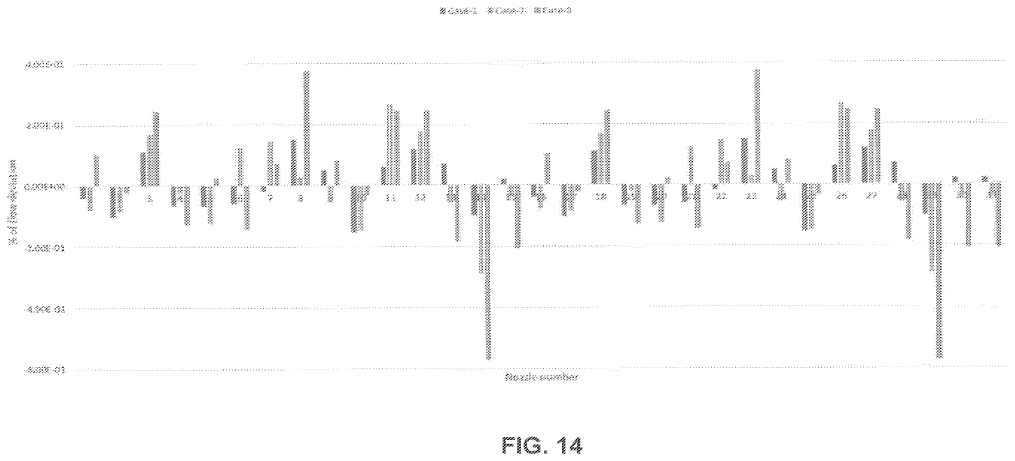

FIG. 13A provides a side view of an applicator 700 under yet another embodiment of the present invention. As can be seen, the FIG. 13A embodiment comprises a substantially disc-shaped body 710 extending from the neck 720 of the applicator 700. The applicator 700 is shown attached to a dispensing device 1200. A bottom portion of the applicator is shown in phantom to reveal a plurality of through openings 750 therein. In this embodiment, the through openings 750 are in the form of nozzles 750 that extend from the body 710 of the applicator 700 and terminate as micropores on the bottom surface 701.

FIG. 13B shows the bottom surface 701 of the FIG. 13A applicator 700. In the pictured embodiment, the applicator 700 comprises 15 individual through-openings.

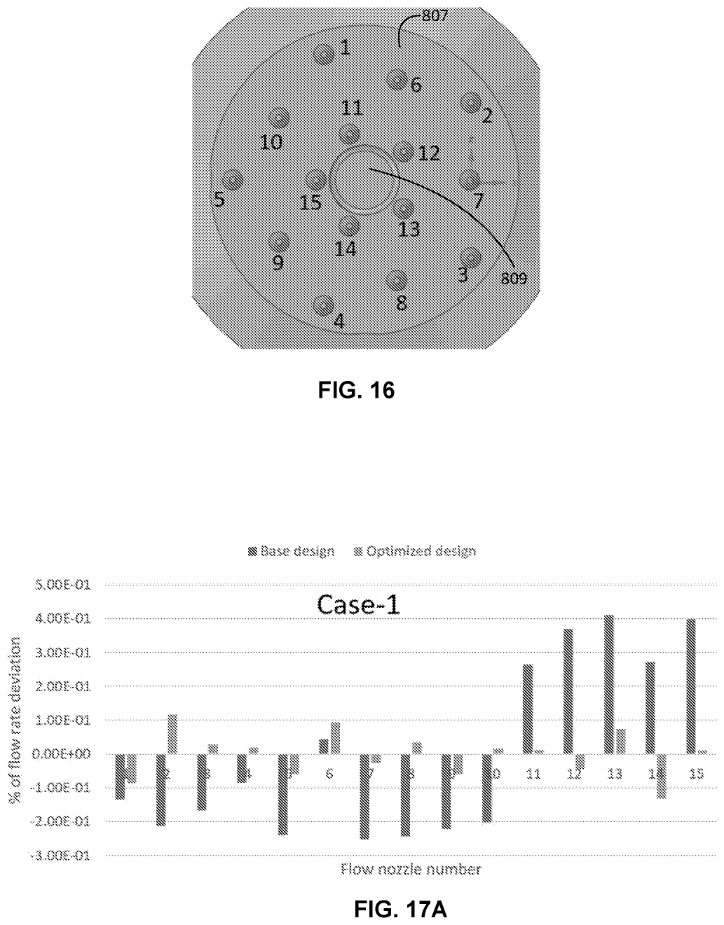

FIG. 16 shows a schematic view taken from within the body and looking down on a bottom portion of an applicator in yet another embodiment. From this perspective, a top side 807 of the bottom portion is visible, and the top of 15 through openings 850 can be seen. In this embodiment, the surface comprises a protrusion 807 that extends from the center up toward the body of the applicator and is configured to assist even distribution of the therapeutic solution to each of the through openings 850.

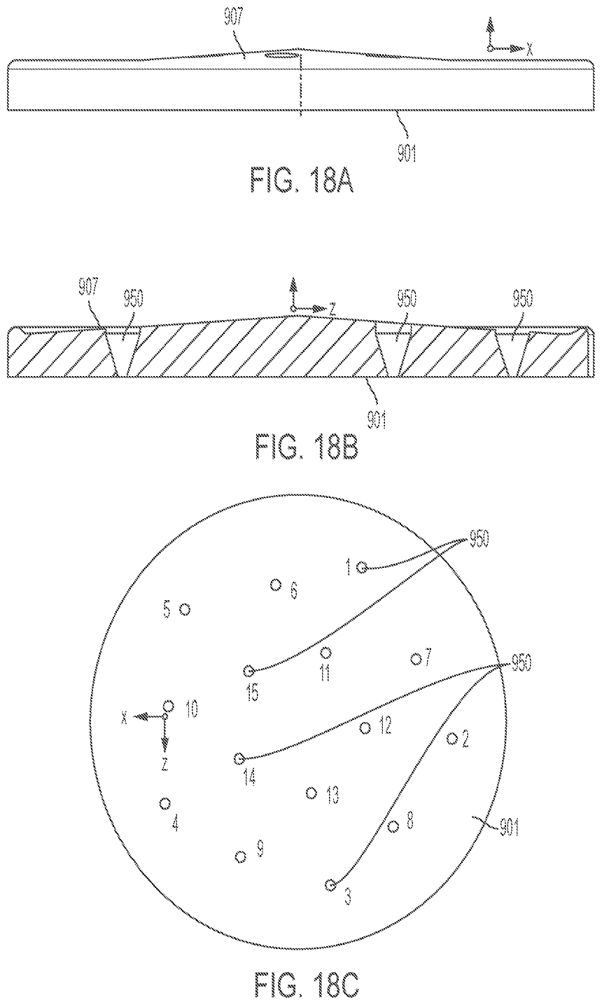

FIG. 18A shows a side view of the bottom portion of an applicator under one embodiment of the present invention. As can be seen, the applicator comprises an inclined face 907 that is configured to permit even distribution of solution flow to each of the through openings (see 950 at FIGS. 18B and C)

FIG. 18B provides a side, cross-sectional view of the FIG. 18A bottom portion. The full length of three through openings 950 if visible as the through openings form micropores on the bottom surface 901 of the applicator.

FIG. 18C shows the bottom surface 901 and micropore configuration of the FIG. 18A embodiment, which is shown with 15 micropores 950.

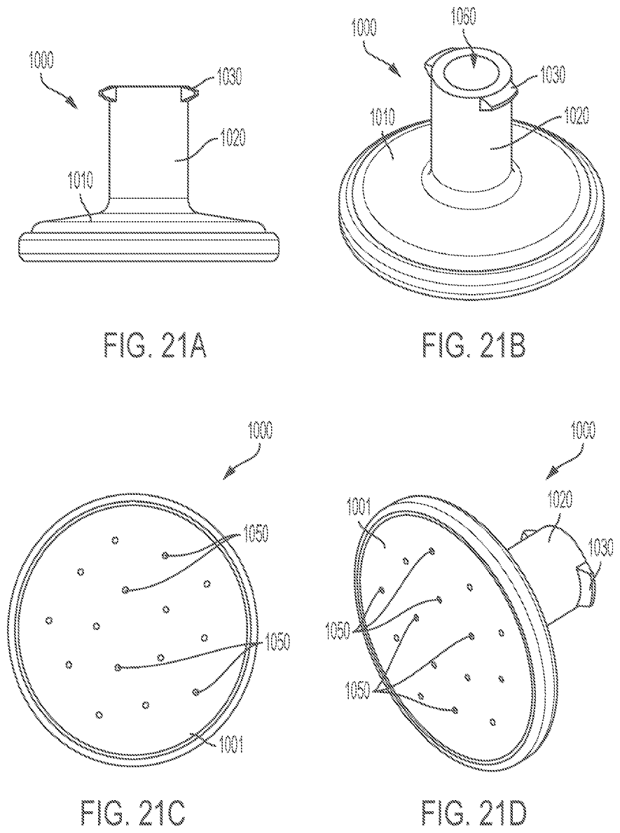

FIG. 21A provides a schematic side view of an applicator 1000 under another embodiment of the present invention. As can be seen, the applicator 1000 includes a means for reversibly attaching the applicator to a dispensing device 1030 at the topmost portion of the applicator 1000. The attachment means 1030 is connected to a widened portion or body 1010 of the applicator via a neck or collar 1020. This embodiment comprises a substantially disc-shaped body 1010 that forms a manifold with at least one through opening (seen more clearly at 1050 of FIGS. 21C & 21D) that terminates in the bottom surface (seen more clearly at 1001 of FIGS. 21C & 21D) of the applicator 1000.

FIG. 21B shows a top perspective view of the FIG. 21A embodiment. This embodiment more clearly shows the lumen 1060 that is continuous throughout the applicator 1000. The lumen 1060 begins within the neck 1020 of the applicator, extends into the body 1010, and is continuous with the one or more through openings 1050 that form micropores on the bottom surface 1001 of the applicator.

FIG. 21C shows the bottom surface 1001 of the applicator 1000 of FIG. 21A. In the FIG. 21C embodiment, the bottom surface 1001 is shown with 15 openings 1050 extending therethrough. In operation, the openings form micropores 1050 that permit controlled application of a solution or ointment when the applicator 1000 is connected to a dispensing device.

FIG. 21D shows a bottom perspective view of the FIG. 21A embodiment.

In embodiments, the attachment means 130, 330, 430, 530, 630 of the applicator 100, 300, 400, 500, 600 can comprise either a female or male Luer fitting. In certain embodiments, the female Luer fitting can comprise a rotating threaded collar that is configured to be screwed onto a male Luer fitting. Alternate embodiments can comprise an attachment means 130, 330, 430, 530, 630 with a tapered fitting that is configured to be joined as a slip-on attachment. In exemplary tapered-fitting embodiments, the attachment means 130, 330, 430, 530, 630 and the dispensing device can be pressed together and reversibly secured via friction. In certain, tapered-fitting embodiments, the attachment mechanism need not comprise threads. Although a Luer-lock fitting is illustrated in FIG. 4, the applicator can be configured to use any attachment means 130 known in the art to be suitable for establishing leak-free or low-leak connections for small-scale fluid transfer. In embodiments, the attachment means 130 can be configured to attach the applicator to any dispensing device known in the art. In embodiments, the dispensing device comprises a container or tube of commercially available topical therapeutic, and the applicator 100, 300, 400, 500, 600 can be configured to attach thereto. In one embodiment, the attachment means 130, 330, 430, 530, 630 of the applicator 100, 300, 400, 500, 600 is configured to be threadably attached to the container or tube of a commercially available topical therapeutic. In certain embodiments, the attachment means comprises a threaded surface that is complementary to the threaded surface of a container opening, wherein the container comprises a commercially available topical therapeutic.

In examples of such threaded embodiments, a lid can first be removed from the threaded nozzle of an off-the-shelf container of topical therapeutic, and the applicator is screwed onto the threaded nozzle of the container. After securing of the applicator thereto, the off-the-shelf container of topical therapeutic can then be squeezed to express the topical therapeutic into the applicator and through the through-openings for application onto a treatment site. The applicator can then be removed. In single-use embodiments, the applicator is discarded after use. In embodiments configured for repeated use, the applicator is sterilized or washed prior to any subsequent use.

In an alternate embodiment, the applicator is integral with the container or tube of commercially available topical therapeutic.

The attachment means 130 an be configured to reversibly tether the applicator 100 to a syringe. In certain embodiments, the attachment means 130 permits tethering of the applicator 100 to a up to a 10-cc syringe. In embodiments, the dispensing device comprises as a 1 to 3-cc syringe, inclusive. The dispensing device can comprise a 1, 2.5, or 5 ml syringe.

In operation of one embodiment, the applicator 100 is attached to a dispensing device filled with a solution. The dispensing device is used to dispense the solution through the opening of the applicator to fill the interior space 160. In filling the interior space 160, the solution flows through the neck or collar 120 of the applicator 100 and into the body that forms a manifold 110. After flowing through the body 110, the solution exits the applicator 100 through at least one micropore 150 for expression onto a tissue of a subject in need thereof

In embodiments, the one or more micropores 150 are substantially flush with the bottom surface 101, and the applicator 100 is devoid of any needles, protrusions, projections, or other extensions.

Although the applicator 100 is shown comprising a conical or disc-shaped body 110 with a circular bottom surface 101, alternate cross-sectional shapes and configurations are also useful. For instance, the bottom surface 101 can be substantially oval or polygonal in shape. In polygonal embodiments, the bottom surface 101 can comprise shapes that are regular or irregular polygons. Embodiments of the applicator 100 bottom surface 101 are substantially triangular in shape, substantially rectangular in shape, or substantially square. Embodiments can comprise straight or curved edges. In still other embodiments, the applicator 100 can comprise a spherical, conical, or disc-like bottom surface 101. The size, shape, or configuration of the bottom surface 101 can vary depending on needs or preferences of the subject or caretaker. The size, shape, or configuration of the bottom surface 101 can also vary with the tissue type, surface area size, wound type, wound size, or tissue location. For instance, disc-shaped embodiments, can be useful for expression of a therapeutic agent onto a large surface area of tissue. After expression, the solution can be configured to reside within, reside upon, or cover the tissue of the subject. In embodiments, the solution is expressed onto the skin, a surface wound, a lesion, or a combination thereof.

The size of the applicator 100 can comprise any size that would be convenient or useful for expression of a therapeutic agent onto any tissue of a subject. In certain embodiments, the bottom surface 101 of the applicator 100 comprises a diameter of up to about 30 mm. The bottom surface 101 can comprise a diameter smaller than about 1 mm. In embodiments, the diameter of the bottom surface 101 ranges from about 1 mm to about 25 mm. The diameter of the bottom surface 101 can range from about 5 mm to about 20 mm. In embodiments, the diameter of the bottom surface 101 is between about 10 mm and 15 mm, inclusive. The diameter of the bottom surface can be about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm.

Additionally, the configuration and number of micropores 150, 350, 450, 550 can vary depending on the needs or preferences of the subject or caretaker. The configuration and number of micropores 150, 350, 450, 550 can also vary with the tissue type, surface area size, wound type, wound size, tissue location, viscosity of the solution carrying the therapeutic agent, or a combination thereof. The applicator 100 can comprise up to fifty micropores 150, 350, 450, 550. In some embodiments, the applicator 100 comprises a single micropore 150, 350, 450, 550. Certain embodiments can comprise up to 100 micropores. Embodiments can comprise between fifteen and sixty micropores, inclusive. Embodiments comprise between one and thirty micropores or openings 150, 350, 450, 550, inclusive. Alternate embodiments can comprise up to twenty micropores 150, 350, 450, 550. Embodiments comprise between five and fifteen micropores 150, 350, 450, 550, inclusive. In embodiments, there can be one, two, three, four, five, six, seven, eight, nine, ten, eleven, twelve, thirteen, fourteen, fifteen, sixteen, seventeen, eighteen, nineteen, or twenty micropores 150, 350, 450, 550. In certain embodiments, the number of micropores 150, 350, 450, 550 is customizable depending the needs or preferences of the subject or caretaker.

The size of each micropore 150, 350, 450, 550 can also vary depending on the needs or preferences of the subject or caretaker. The configuration and number of micropores 150, 350, 450, 550 can vary with the tissue type, surface area size, wound type, wound size, tissue location, viscosity of the solution carrying the therapeutic agent, therapeutic agent to be expressed, the size of the syringe employed, or a combination thereof (see Clayton and Willihnganz, Basic Pharmacology for Nurses, pp 120, 135-36 ( 17^{th} ed. 2017)). The size of the micropores can comprise any size that is sufficient to permit controlled application of a solution. The size of the micropores can comprise any diameter that is sufficient to permit controlled and unobstructed application of a hyper-viscous fluid therethrough. The micropores can comprise a size that is sufficient to permit a solution with a viscosity similar to that of petroleum jelly to flow therethrough. In embodiments, the size of each micropore 150, 350, 450, 550 can be as large as the inner diameter of a 15-gauge needle or as small as the inner diameter of a 33-gauge needle. The diameter of each micropore 150, 350, 450, 550 can be as large as that of a 25-gauge needle or as small as that of a 32-gauge needle. The size of at least one micropore 150, 350, 450, 550 can be substantially the same as the inner diameter of a 33-gauge, 32-gauge, 31-gauge, 30-gauge, 29-gauge, 28-gauge, 27-gauge, 26gauge, or 25-gauge needle. At least one micropore 150, 350, 450, 550 can comprise a diameter of up to about 2 mm. In embodiments, the diameter of at least one micropore 150, 350, 450, 550 is less than about 1.5 mm. The diameter of at least one micropore 150, 350, 450, 550 can be as smaller than 0.01 mm. In embodiments, at least one micropore 150, 350, 450, 550 comprises a diameter between about 0.005 mm and about 0.50 mm. At least one micropore 150, 350, 450, 550 can comprise a diameter of between about 0.01 and about 0.3 mm. In embodiments, at least one micropore 150, 350, 450, 550 comprises a diameter of between about 0.09 and about 0.20 mm. In certain embodiments, the diameter of at least one micropore is about 0.05 mm, about 0.06 mm, about 0.07 mm, about 0.08 mm, about 0.09 mm, about 0.10 mm, about 0.11 mm, about 0.12 mm, about 0.13 mm, about 0.14 mm, about 0.15 mm, about 0.16 mm, about 0.17 mm, about 0.18 mm, about 0.19 mm, or about 0.20 mm. The size of at least one micropore 150, 350, 450, 550 can be configured to prevent any structural or function disruption of a material within the therapeutic agent. In one embodiment, the size of at least one micropore 150, 350, 450, 550 is configured to prevent structural or functional disruption of platelets. In embodiments, each of the micropores 150, 350, 450, 550 are substantially identical in size. In alternate embodiments, the size of each of the micropores 150, 350, 450, 550 varies.

In embodiments, the solution administered comprises more than one type of therapeutic agent. The solution administered can comprise between one and ten different therapeutic agents. In embodiments, the solution administered can comprise between two and five different therapeutic agents. The solution can comprise two, three, four, five, six, seven, eight, nine, or ten different therapeutic agents. In one embodiment, the solution administered comprises a single type of therapeutic agent. The solution administered can comprise a topical solution or can comprise a solution to be administered upon or within diseased, wounded, injured, burned, or otherwise comprised tissue.

The therapeutic agents can comprise biological fluids, pharmaceutical compositions, or any other therapeutic compound known by one of skill in the art. Pharmaceutical compositions can comprise hydrophobic agents, hydrophilic agents, or a combination of both. In one embodiment, the therapeutic agent promotes wound healing. Examples of such wound-healing agents include, but are not limited to an antibiotic, an antihistamine, an anti-inflammatory agent, a coagulant, a steroid, an anti-fungal agent, an angiogenic compound, an analgesic, a biofilm inhibitor, a topical chemotherapeutic, a corticosteroid, tissue regenerative agents, or a combination thereof. The therapeutic agent can further comprise bioactive molecules such as growth hormones or growth factors. Such growth factors include, but are not limited to epidermal growth factor, transforming growth factor, vascular endothelial growth factor, fibroblast growth factor, platelet-derived growth factor, interleukins, colony-stimulating factors, keratinocyte growth factor, or a combination thereof. The therapeutic agent can also comprise broad spectrum antimicrobials such as chlorhexidine gluconate. The therapeutic agent can comprise an antimicrobial, such as an antiviral agent, an antibiotic, an anti-fungal agent, or a combination thereof. Examples of potential antibiotics include, but are not limited to, clindamycin or vancomycin. In embodiments, the antibiotic is culture-specific. The antifungal agent can be any appropriate antifungal agent including, but not limited to, nystatin. Non-limiting examples of the anti-inflammatory agent include betamethasone, hydrocortisone, corticosterone, prednisone, a combination thereof. The anti-inflammatory can also comprise a nonsteroidal anti-inflammatory (NSAID) drug such as aspirin, ibuprofen, celecoxib, ketorolac, or any other appropriate NSAID. In embodiments with a coagulant, the coagulant can comprise thrombin, prothrombin, avitene, or any other appropriate coagulant. The topical chemotherapeutic can include fluorouracil, imiquimod, diclofenac, ingenol mebutate, or any other topical chemotherapeutic or combination of topical chemotherapeutic known to those of skill in the art. Biological fluids can comprise any a fluid with any biological material known to be useful in a medical or cosmetic procedure. Biological fluids can comprise biological material that is autologous, homologous, heterologous, or a combination thereof. In embodiments, biological fluid comprises blood, serum, leukocyte-rich plasma, stem cells, a platelet concentrate, or a combination thereof. In embodiments, platelet-rich concentrates comprise platelet-rich plasma (PRP), platelet-rich fibrin (PRF), or a combination thereof. PRP can be acquired through following collection and centrifugation of a subject's blood, which separates the blood into multiple layers. In certain embodiments, the subjects blood undergoes at least two rounds of centrifugation such that the blood is separated into a platelet poor plasma layer, a PRP layer, and a red blood cell layer. After centrifugation, the PRP layer can be withdrawn into a syringe and used for administration.

In various exemplary embodiments, the applicator 100 can be reusable. In reusable embodiments, the applicator 100 can be sterilized or can be sterile when provided or obtained. By way the example, the applicator can be configured to withstand autoclave sterilization, chemical sterilization, or a combination thereof. In alternate embodiments, the applicator 100 can be configured for a single use. The applicator 100 can be disposable.

The applicator can be comprised of any material currently known by those of skill in the art or later developed that is suitable for use in medical or cosmetic procedures. In embodiments, the applicator is comprised of a medical-grade material. The applicator can be comprised of a medical grade polymer, metal, or a combination thereof. In certain embodiments, the applicator is comprised of surgical metal. The applicator can be comprised of stainless steel, titanium, tantalum, gold, platinum, palladium, or any other metal or combination of metals suitable for surgical use.

In one embodiment, the applicator comprises a surgical-grade, stainless steel disc with micropores attached to a cap and Luer-lock hub made of plastic at the top portion to attach to a syringe.

In embodiments, the applicator 100 is integral with a dispensing device. The applicator 100 can be integral with a syringe.

Another aspect of the present invention includes a method of administering one or more solutions by use of an applicator 100 in accordance with any embodiment disclosed within this specification or otherwise apparent from the descriptions herein. In one embodiment, the method includes obtaining or providing a volume of a solution comprising a therapeutic agent and expressing a volume of solution through the applicator 100 onto or into the tissue of a subject in need thereof.

In embodiments, the therapeutic agent is obtained by drawing a required volume of a therapeutic solution into a sterile syringe. The method can further include removal of the needle followed by installation of the applicator 100. Following instillation of the applicator 100, the desired volume of therapeutic can be administered through the applicator 100 by depression of a syringe plunger. In embodiments the therapeutic solution can be disposed within a syringe before being obtained.

The therapeutic solution can be obtained by withdrawing a volume of biological fluid from a subject. The biological fluid can then be applied through the applicator 100 onto a patient or subjected to further processing. In certain embodiments, further processing can include isolation of a therapeutic agent from the biological fluid. Isolation can comprise any means known in the art. In embodiments, isolation of the therapeutic agent is accomplished through centrifugation.

Following administration of the therapeutic agent, the applicator 100 can be removed and sterilized if the applicator 100 is reusable. In disposable embodiments, the applicator 100 can be removed for the syringe and discarded or discarded with the syringe.

Another aspect of the present invention comprises a kit that further includes an applicator 100 in accordance with any embodiment disclosed within this specification or otherwise apparent from the descriptions herein. In embodiments, the kit comprises the applicator 100 and instructions for use. The instructions can be physically provided with the kit or accessible separately from the kit, such as via the retailer's or manufacturer's website. In embodiments, the kit includes a dispensing device, a therapeutic agent, or a combination thereof. In certain embodiments, the dispensing device can include a syringe that is pre-loaded with a particular therapeutic agent. The kit can include an empty syringe. In embodiments, the applicator may be integral with a syringe or other dispensing device.

The kit can include a syringe, a needle, an applicator, or a combination thereof. Kits that comprise a syringe and needle are useful for embodiments wherein the therapeutic agent comprises a biological fluid.

In certain kit embodiments, the kit comprises applicators of a variety of sizes and shapes from which the subject, patient, physician, nurse, clinician, beautician, or other caretaker can select as deemed appropriate. Such embodiments permit the kit recipient to tailor the applicator 100 to the needs or preferences of the subject or caretaker. In embodiments, the therapeutic agent can be provided with the kit or can be provided or obtained separately therefrom. Kit embodiments without a therapeutic agent allow the caretaker or subject to select an appropriate therapeutic agent for expression through the applicator 100.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Safe and Controlled Application of Platelet-Rich Plasma, Stem Cells, and Other Serums to Wounded Skin Using a New Applicator

Background: Platelet-rich plasma (PRP), stem cells, peptides, and other serums are frequently applied to wounded skin to enhance wound healing. Controlled, intentional wounds are often created in clinical practice with procedures such as microneedling or fractional laser resurfacing. Other wounds on skin or mucosal surfaces may result from injury, trauma, or illness. Often in clinical practice, serums such as PRP, stem cells, or peptides are applied via open ended syringes. In this method, the syringe hovers over the tissue while the product is dripped onto the surface which does not allow for controlled application of the product onto the wounded skin. Another method involves dripping the product from a needle attached to the syringe which poses an unwanted risk of needle stick to the patient and provider.

Objective: To describe the use of a device for the sterile, safe, and controlled application of serums to wounded skin.

Material and Methods: A simple applicator comprising a smooth, circular disc permeated with multiple micropores on a Luer lock base to attach to any standard Luer lock syringe was designed. The applicator can be made of medical grade polymer with a smooth interface and simple, easy to use design in order to be supplied as a sterile, single use only applicator.

Results: When attached to any standard Luer lock syringe, the applicator allows for the sterile, controlled application of a variety of serums to wounded skin.

Conclusion: A simple applicator has been designed to allow for the safe, sterile, and controlled application of serums to wounded skin. This sterile device can be attached to any standard Luer-lock syringe to aid in the needle-free and hands-off application of a variety of serums onto wounded skin. The device can be single-use or designed for re-use. The device is practical and requires no additional supplies or training to implement into clinical practice. This device meets an unmet need in the application of serums or other products to wounded tissue.

Introduction: Platelet-rich plasma, stem cells, peptides, and other serums are frequently applied to wounded skin to enhance wound healing. Wounds are often created in clinical practice with procedures such as microneedling or fractional laser resurfacing, or wounds may result from injury, trauma, or illness from diseases such diabetes mellitus and peripheral vascular disease. Often serums such as PRP, stem cells, and peptides are applied to wounded tissue via an open-ended syringe which does not allow for controlled application and can create splatter of the product. For example, with blood borne products, such as PRP, splatter of product poses an infectious disease risk if the product comes in contact with the eyes or mucous membrane of the clinician. With this method the serum is often spread by the clinician over the tissue with a non-sterile, gloved hand which could contaminate or affect the viability of the product. Another common method of application of serum involves expressing the product through a needle attached to a syringe. This frequently used method carries a significant risk of unwanted needle stick to the patient and practitioner. This article describes a simple applicator that when attached to any universal Luer lock syringe allows for the safe, controlled, sterile application of PRP, stem cells, peptides, or other serums to wounded tissue.

Materials and Methods: An applicator with a circular, smooth interface permeated with multiple micropores on a universal Luer lock base to attach to any Luer lock syringe was designed. The sterile device when attached to a syringe allows the clinician to control the application of the serum by controlling the pressure on the syringe. The disc can hover slightly above the surface while with controlled pressure on the syringe the practitioner expresses the product onto tissue. Direct contact between the wounded skin and the smooth, medical grade polymer interface on the device can also be employed to apply serum in a gliding motion directly onto tissue. In both application methods, the applicator allows the practitioner to have safe, controlled application of serum without risk of splatter, contamination, or needle-stick as compared to other methods.

Discussion: Many studies and articles describe the use of platelet-rich plasma, stem cells, peptides, and other serums to wounded skin and other tissues to enhance wound healing. In clinical practice, these serums are applied in an uncontrolled, non-sterile manner by expressing the product from an open-ended syringe onto the tissue, then using a non-sterile gloved hand to spread the serum over the wounded tissue. Limitations of this method include risk of splatter when the serum is expressed from the open-ended syringe, contamination of the product of spread with non-sterile gloved hands, and lack of control of application of the product. Another commonly used method for applying serum to wounded skin is the expression of the serum from a needle attached to the syringe containing the product. Although this allows for more control of the speed and more precision in the application of the serum as it is expressed from the syringe, this method poses a high risk for unwanted needle-stick for the patient and the provider, making it a high-risk application technique.

To address these limitations and for the practical use in our clinical settings, a simple, sterile, safe applicator was designed that could easily be used for the controlled application of PRP, stem cells, peptides, or other serum onto wounded skin. No special kits or additional equipment other than standard Luer lock syringes are needed to use the new applicator device. The device can be a circular disc permeated with multiple micropores with a Luer lock base. Different sized disc can be manufactured for a variety of sizes to accommodate for different size surface areas being treated. The device is made from a disposable, medical grade polymer packaged as a sterile, single use only applicator. The clinician can choose from or combine two methods of application using the device. The first is to hover over the wounded tissue while applying controlled pressure on the syringe to apply serum without contact to the tissue. The second method is gentle contact of the smooth interface of the applicator disc touching the tissue to spread serum while being expressed from the applicator with light pressure on the syringe. In both methods, the serum is safely applied to the wounded tissue.

The benefits of the applicator include ease of use, safe application without risk of needle-stick, controlled application methods without risk of aerosol inhalation or uncontrolled splatter, sterile without contamination or adulteration of serums, and compatible with standard Luer lock syringes which requires no further purchase of specialty products which increase overhead costs.

Conclusion: Safe, sterile, controlled application of serum to wounded skin can be achieved using a simple new applicator that attaches to standard Luer lock syringes currently used in practices performing these procedures or clinics treating wounds. The device is practical and requires no additional supplies or training to implement into clinical practice. This device meets an unmet need in the application of serums or other products to wounded tissue.

### Example 2

Flow deviations were determined for each of the micropores (or nozzles) in three separate embodiments of the present invention. Table 1 summarizes the test conditions used for assessment of flow deviation. Briefly, four different embodiments were tested under three separate conditions or “cases.” In case 1, water was passed through each embodiment at a flow rate of 0.5 ml per second. In case 2, water was passed through each embodiment at a rate of 1 ml per second. Finally, in case 3, petroleum jelly was warmed to 100° C. and passed through each embodiment at a flow rate of 0.5 ml per second.

TABLE 1
Experimental groups for Flow Deviation Assessment of

| | | Density | Viscosity | Flow |
|---|---|---|---|---|
| Cases | Fluid | (kg/m3) | (Pa − s) | (ml/sec) |
| Case-1 | water | 1000 | 0.001 | 0.5 |
| Case-2 | water | 1000 | 0.001 | 1 |
| Case-3 | petroleum jelly | 830 | 0.0151 | 0.5 |
| | at 100° C. | | | |

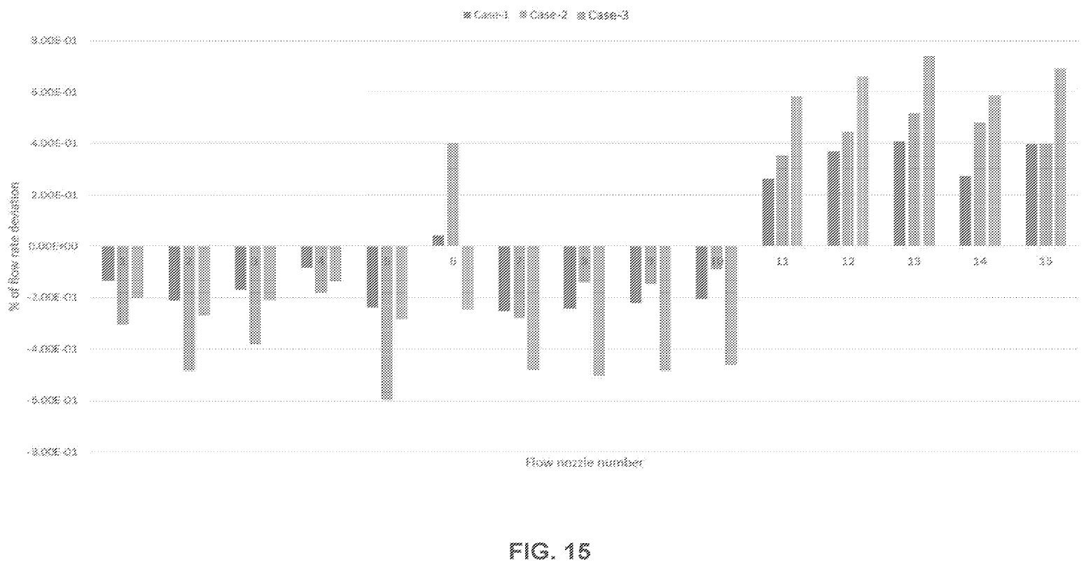

FIG. 14 provides a graphical representation of flow deviation through each of the micropores of the FIG. 12 embodiment, and FIG. 15 provides a graphical representation of flow deviation through each of the micropores of the FIG. 13 embodiment. Measurements were obtained in the three cases described in Table 1, and each numeral represents a different micropore or nozzle of the embodiment tested. As can be seen, flow deviations in the FIG. 12 embodiment are more random than the FIG. 13 embodiment, indicating that the FIG. 13 embodiment is better than the FIG. 12 embodiment for a wide range of push pressure and fluid viscosity. As can be seen, flow deviation from mean value increases with increase in pushing pressure (total dispense rate). Deviation also increases for thicker fluid e.g. petroleum jelly. The deviation pattern also appears random. Further improvements include the embodiments of FIGS. 16 and 18, which are designed to provide increased control in flow rate over a range of application and to minimize deviations.

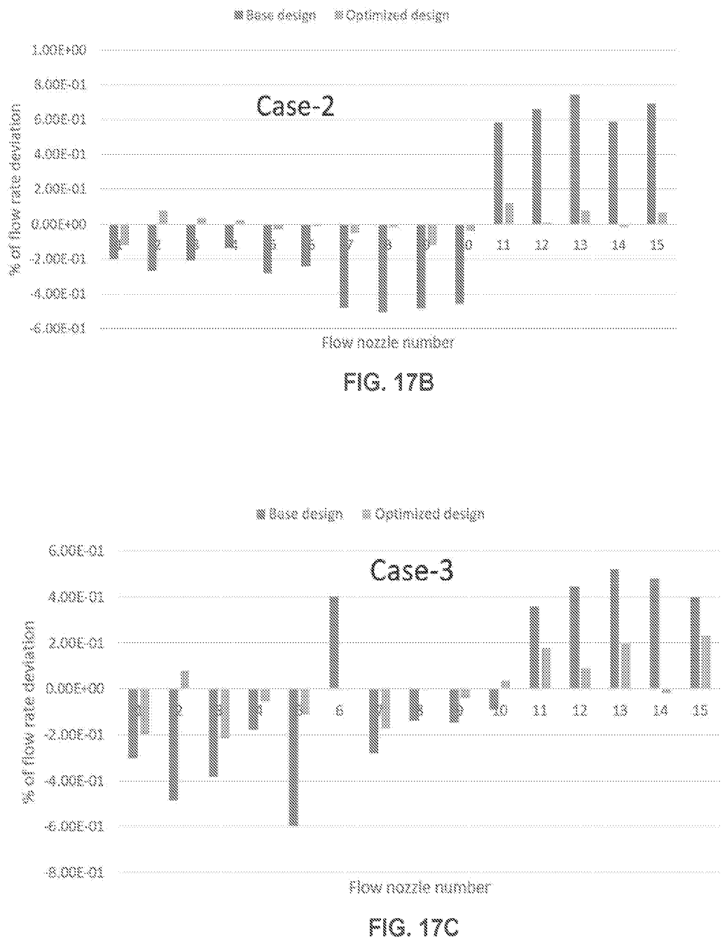

FIG. 17A provides a graphical representation of flow rate deviation through each of the micropores of the FIG. 16 embodiment as compared to the FIG. 12 embodiment under the conditions of case 1 (as detailed in Table 1). FIG. 17B provides a graphical representation of flow rate deviation through each of the micropores of the FIG. 16 embodiment as compared to the FIG. 12 embodiment under the conditions of case 2 (as detailed in Table 1). FIG. 17C provides a graphical representation of flow rate deviation through each of the micropores of the FIG. 16 embodiment as compared to the FIG. 12 embodiment under the conditions of case 1 (as detailed in Table 1). Each numeral corresponds to the applicable micropore or nozzle of FIG. 16.

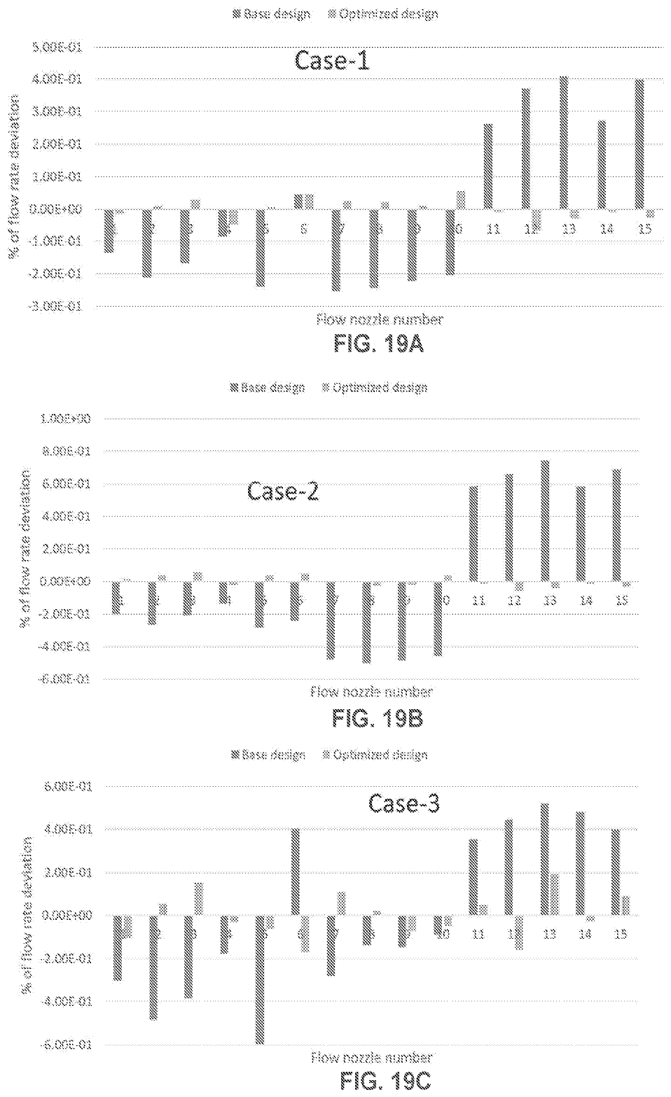

FIG. 19A provides a graphical representation of flow rate deviation through each of the micropores of the FIG. 18 embodiment as compared to the FIG. 12 embodiment under the conditions of case 1 (as detailed in Table 1). FIG. 19B provides a graphical representation of flow rate deviation through each of the micropores of the FIG. 18 embodiment as compared to the FIG. 12 embodiment under the conditions of case 2 (as detailed in Table 1). FIG. 19C provides a graphical representation of flow rate deviation through each of the micropores of the FIG. 18 embodiment as compared to the FIG. 12 embodiment under the conditions of case 3 (as detailed in Table 1). Each numeral corresponds to the applicable micropore or nozzle of FIG. 18C.

As can be seen, as compared to the FIG. 12 embodiment, the embodiments of FIGS. 16 and 18 provide a significantly reduced flow deviation as compared to the FIG. 12 embodiment for all the cases (normal dispense rate, higher dispense rate and thicker fluid).

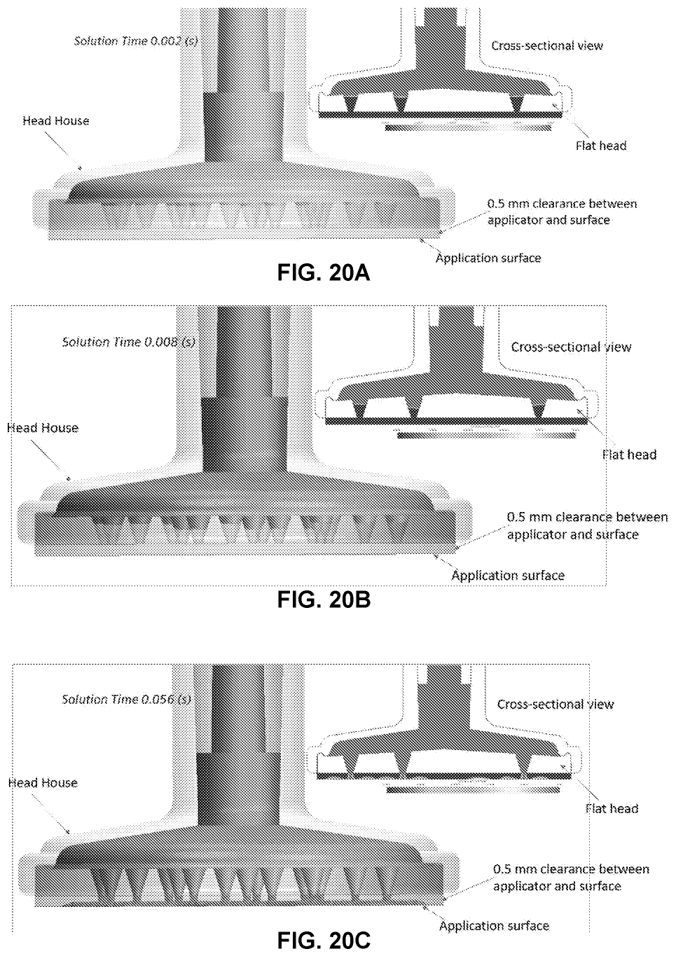

FIG. 20A provides a graphical snapshot of fluid dynamics at 2 milliseconds following initiation of water dispersion at a flow rate of 0.5 ml/second (T=2 ms). In the large, central portion of the figure, the applicator and through openings (also referred to herein as “nozzles”) are shown in phantom and water is shown filled within the applicator neck and body. The inset provides a cross-sectional view of the applicator at T=2 ms.

FIG. 20B shows graphical snapshot of fluid dynamics at about 8 milliseconds following initiation of water dispersion at a flow rate of 0.5 ml/second (T=8 ms). In the large, central portion of the figure, the applicator and nozzles are shown in phantom and fluid is shown passing from the body and into the nozzles. The inset provides a cross-sectional view of the applicator at T=8 ms.

FIG. 20C shows graphical snapshot of fluid dynamics at about 56 milliseconds following initiation of water dispersion at a flow rate of 0.5 ml/second (T=56 ms). In the large, central portion of the figure, the applicator and nozzles are shown in phantom and fluid is shown passing exiting the nozzles of the applicator. The inset provides a cross-sectional view of the applicator at T=56 ms.

### Example 3

Indications for Use:

It is recommended that the RX: Applicare is used to apply serum, fluids, and other wound healing products dispersed from a syringe onto wounded tissue for needle-free, hands-free, controlled and sterile application.

Instructions for Use:

After opening the pouch, remove the sterile, single use only RX: Applicare device and attach securely via the Luer lock hub to a compatible Luer lock syringe. Dispense fluid, serum, or other product from the syringe either directly onto tissue or by hovering over tissue by applying gently pressure to the syringe. The RX:Applicare may be used to spread the product over the tissue while also dispensing the product from the syringe onto tissue. After completing the application, dispose of the RX:Applicare in appropriate medical waste container. This example is intended for single use only.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. A device for controlled application of a therapeutic solution to a tissue of a subject in need thereof, comprising:
an applicator that further comprises at least one micropore, wherein
the at least one micropore comprises an opening that is substantially flush with a bottom surface of the applicator; and
a means for reversibly attaching the applicator to a dispensing device.

2. The device of claim 1, comprising a plurality of micropores, wherein each of the micropores is substantially identical in size.

3. The device of claim 1, wherein the therapeutic solution comprises a topical solution.

4. The device of claim 1, wherein the device is configured for use with a cosmetic procedure, a medical procedure, or a combination thereof.

5. The device of claim 4, wherein the medical procedure comprises cancer treatment, lesion treatment, pain control, wound care, burn care, skin care, or a combination thereof.

6. The device of claim 4, wherein the cosmetic procedure comprises treatment of skin conditions, treatment of skin damage, skin resurfacing, skin rejuvenation, or a combination thereof.

7. The device of claim 1, wherein the dispensing device comprises a syringe.

8. The device of claim 7, wherein the means for attaching the applicator to the syringe comprises a Luer fitting.

9. The device of claim 1, wherein the dispensing device comprises a container of commercially available topical therapeutic agent, and the therapeutic solution comprises the contents of the container of commercially available topical therapeutic agent.

10. The device of claim 9, wherein the means for reversibly attaching the applicator to the container of commercially available topical therapeutic agent comprises
a first threaded surface disposed on a neck of the applicator,
wherein the first threaded surface comprises threads that are complementary to a second threaded surface that is disposed near an opening of the container of commercially available topical therapeutic agent.

11. The device of claim 1, wherein the therapeutic solution comprises a therapeutic agent.

12. The device of claim 11, wherein the therapeutic agent comprises a topical cream.

13. The device of claim 12, wherein the topical cream comprises an antibiotic, an analgesic, a steroid, an anti-fungal agent, or a combination thereof.

14. The device of claim 11, wherein therapeutic agent comprises a pharmaceutical composition, a biological fluid, or a combination thereof.

15. The device of claim 14, wherein the biological fluid is autologous, homologous, heterologous, or a combination thereof.

16. The device of claim 14, wherein the biological fluid comprises serum, stem cells, a platelet concentrate, or a combination thereof.

17. The device of claim 16, wherein the platelet-rich concentrate comprises platelet-rich plasma (PRP), platelet-rich fibrin (PRF), or a combination thereof.

18. The device of claim 1, wherein the diameter of the micropore is at least the size of the inner diameter of a 33-gauge needle.

19. The device of claim 1, wherein the micropore comprises a diameter that is at least about 0.1 mm.

20. The device of claim 1, wherein the micropore comprises a diameter that is less than about 0.5 mm.

21. The device of claim 20, wherein the micropore comprise a diameter of about 0.1 to about 0.2 mm.

22. The device of claim 1, wherein the micropore comprises a diameter of about 1 mm or less.

23. The device of claim 2, wherein the plurality of micropores comprises at least four micropores.

24. The device of claim 2, wherein the plurality of micropores comprises up to 20 micropores.

25. The device of claim 1, wherein the bottom surface of the applicator is substantially circular, substantially oval, or substantially polygonal in shape.

26. The device of claim 25, wherein the applicator is substantially disc-shaped.

27. The device of claim 1, wherein the applicator comprises a medical-grade material.

28. The device of claim 27, wherein the applicator comprises a surgical metal, a medical-grade polymer, or a combination thereof.

29. The device of claim 28, wherein the surgical metal comprises stainless steel, titanium, tantalum, gold, platinum, palladium, or a combination thereof.

30. The device of claim 1, wherein the tissue comprises skin, muscle, connective tissue, or a combination thereof.

31. The device of claim 30, wherein the tissue comprises wounded, fractionated or resurfaced skin.

32. The device of claim 30, wherein the tissue comprises dermis, epidermis, subcutaneous fat, fascia, or a combination thereof.

33. The device of claim 1, wherein the device is reusable.

34. The device of claim 1, wherein the device is disposable.

35. A device for controlled application of a platelet concentrate during a cosmetic resurfacing procedure, the device comprising:
a disc-shaped applicator further comprising at least 12 micropores, wherein
each of the micropores comprise a diameter of 1 mm or less;
each of the micropores comprises an opening that is substantially flush with a bottom surface of the applicator; and
a Luer fitting.

36. The device of claim 35, wherein the platelet-rich concentrate comprises platelet-rich plasma (PRP), platelet-rich fibrin (PRF), or a combination thereof.

37. A method of administering one or more therapeutic solutions to a tissue of a subject in need thereof, the method comprising:
obtaining a volume of therapeutic solution; and
expressing the volume of therapeutic solution through the device of any one of claims 1-36 and onto the tissue of the subject.

38. The method of claim 37, wherein
the therapeutic solution comprises a biological fluid; and
obtaining the volume of therapeutic solution comprises drawing the biological fluid into a syringe.

39. The method of claim 38, further comprising attaching the applicator to the syringe.

40. The method of claim 37, wherein
the therapeutic solution comprises a topical therapeutic agent; and the dispensing device comprises a container of commercially available topical therapeutic agent.

41. The method of claim 40, further comprising attaching the applicator to the container of commercially available topical therapeutic agent.

42. The method of claim 37, wherein the tissue comprises dermis, epidermis, subcutaneous fat, fascia, or any combination thereof.

43. A method of reducing contamination during application of a topical solution to a subject in need thereof, the method comprising:
obtaining a volume of topical solution; and
expressing the volume of topical solution through the device of any one of claims 1-36 and onto the tissue of the subject.

44. A kit for applying a therapeutic solution comprising the device of any one of claims 1-36 and instructions for use.

45. The kit of claim 44, further comprising a sterile syringe, a therapeutic agent, or a combination thereof.

46. A kit for treating or restoring a tissue of a subject comprising the device of any one of claims 1-36 and instructions for use.

47. A kit for use with cosmetic resurfacing procedures comprising the device of any one of claims 1-36 and instructions for use.
